(19) United States
(12) Patent Application Publication
(10) Pub. No.: US 2015/0065954 A1
(43) Pub. Date: Mar. 5, 2015

(54) NOVEL ENHANCED BALLOONS HAVING PRE-STRESSED STRUT REINFORCEMENT

(71) Applicant: TELEFLEX MEDICAL INCORPORATED, Research Triangle Park, NC (US)

(72) Inventor: Jeffrey Vitullo, Reading, PA (US)

(21) Appl. No.: 14/385,365

(22) PCT Filed: Mar. 11, 2013
(86) PCT No.: PCT/US13/30180

§ 371 Date: Sep. 15, 2014

Related U.S. Application Data
(60) Provisional application No. 61/611,186, filed on Mar. 15, 2012.

(51) Int. Cl. A61M 25/10

(52) U.S. Cl. CPC A61M 25/1002; A61M 25/104; A61M 2025/1084
USPC 604/103.07

(57) ABSTRACT

A medical device system with pressurized balloon having a radial wrap configuration yields improved withdrawal force, and has pre-stressed struts for reinforcing at fold points for ease of inflation, deflation, and heightened radiopacity.

### RELATED APPLICATIONS

This application claims the full Paris Convention benefit of and priority to U.S. provisional patent application Ser. No. 61/611,186, the contents of which is incorporated by this reference as if fully set forth herein in its entirety.

### BACKGROUND OF THE DISCLOSURE

The present invention relates to medical devices. In particular, the instant teachings relate to systems for lumenal/vascular access to impart treatment to mammals.

### FIELD OF THE DISCLOSURES

Pressurized balloons have plethoric uses within the medical field. Of prime concern, in salient applications of the instant systems are the dueling issues of low insertion profile, and ease of deployment. According to embodiments, each of these longstanding needs is addressed.

### OBJECTS AND SUMMARY OF THE DISCLOSURE

Briefly stated, a medical device system with pressurized balloon having a radial wrap configuration yields improved withdrawal force, and has pre-stressed struts for reinforcing at fold points for ease of inflation, deflation, and heightened radiopacity.

According to embodiments, there is provided a PTA balloon having pre-stressed struts for reinforcing a spiral wrapped balloon.

According to embodiments, the reinforcing struts are pre-stressed to return to folding points upon retraction into delivery sheaths

According to embodiments, reinforcing struts expand at inflation to unwrap balloons evenly, provide reinforcement and increase radiopacity.

According to embodiments, there is disclosed a medical device system, comprising, in combination, a pressurized balloon adopted for inflation and deflation between an expanded diameter and a collapsed configuration, wherein mounting of the balloon on a catheter system is defined by a spiral wrap of the balloon during the collapsed configuration and, the balloon is equipped with struts for reinforcement, whereby the struts are at fold points and aid in the spiral folding and unfolding, allowing more even inflation and deflation of the balloon.

### BRIEF DESCRIPTION OF THE FIGURES

FIG. 1 is a schematic of a radially wrapped balloon;

FIG. 2 is a schematic of a radially wrapped balloon;

FIG. 3 is a schematic of pre-stressed reinforcing struts.

### DETAILED DESCRIPTION OF THE INVENTIONS

The present inventor has discovered that radially wrapping medical device balloons and reinforcing the same with pre-stressed struts enables better functionality, including strength, radiopacity, burst pressure and shelf life.

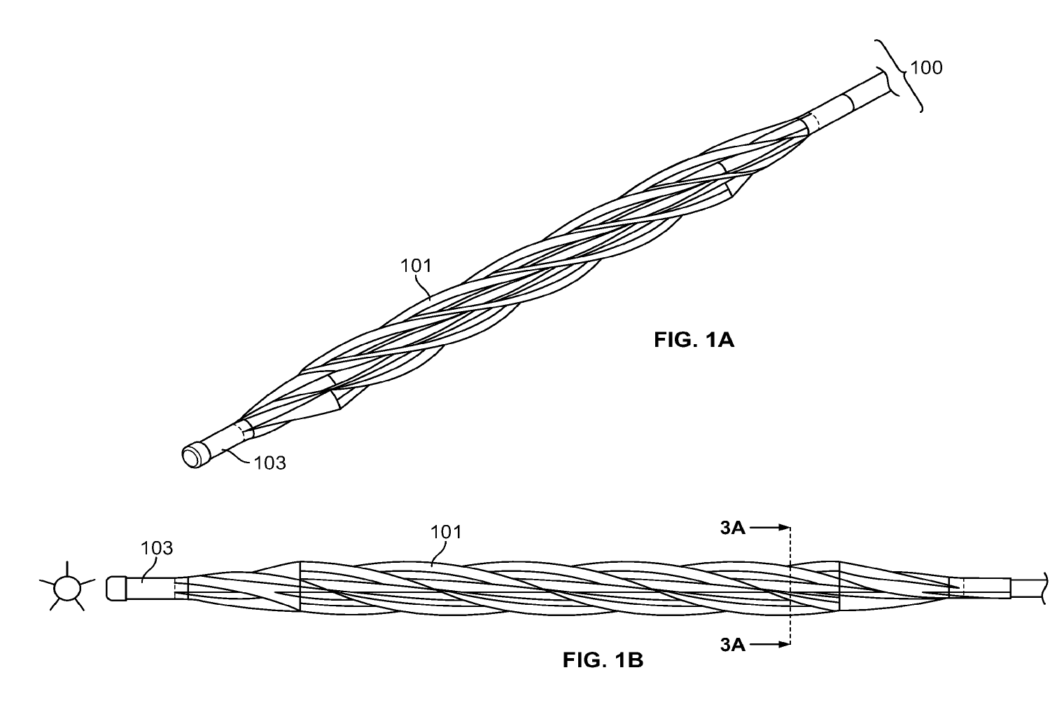

Referring now to FIG. 1-FIG. 3, those skilled in the art understand catheter emplacement, the Seldinger technique and required system elements.

The first figure shows a schematic illustration of system 100 (shown in isolation on balloon segments, the introducer set and all functional elements expressly incorporated herein by reference from U.S. Letters Patents Nos. 5,015,230; 4,362,150; 5,318,587; 5,853,389; 7,597,702; 7,828,767 and 6,013,055) showing the radially collapsed configuration of balloon 101, allowing for insertion into vascularly challenged spaces via proximal end 103.

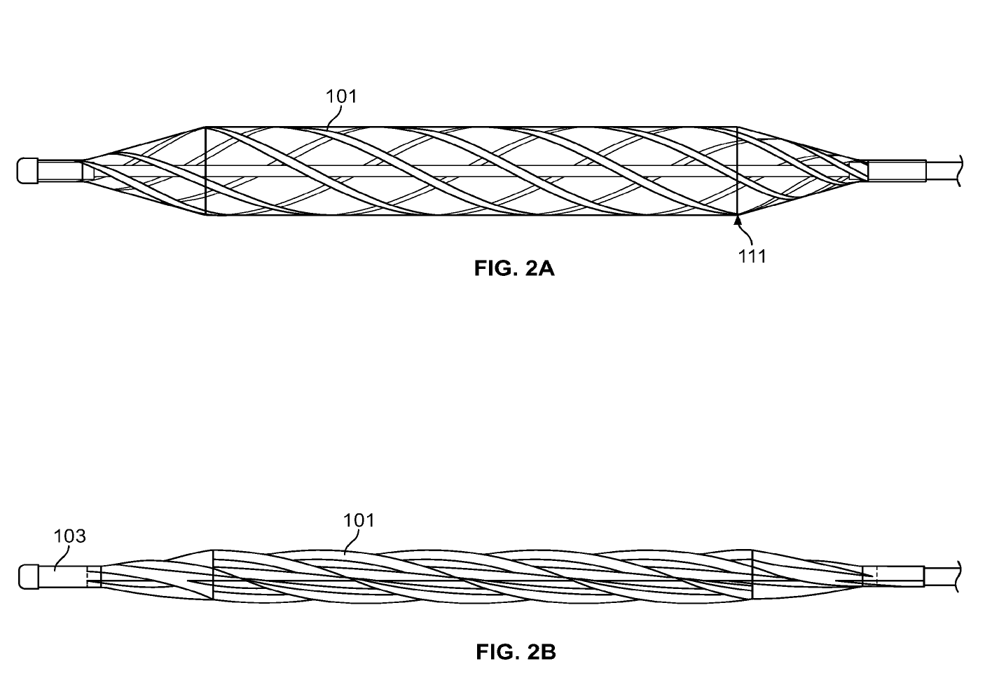

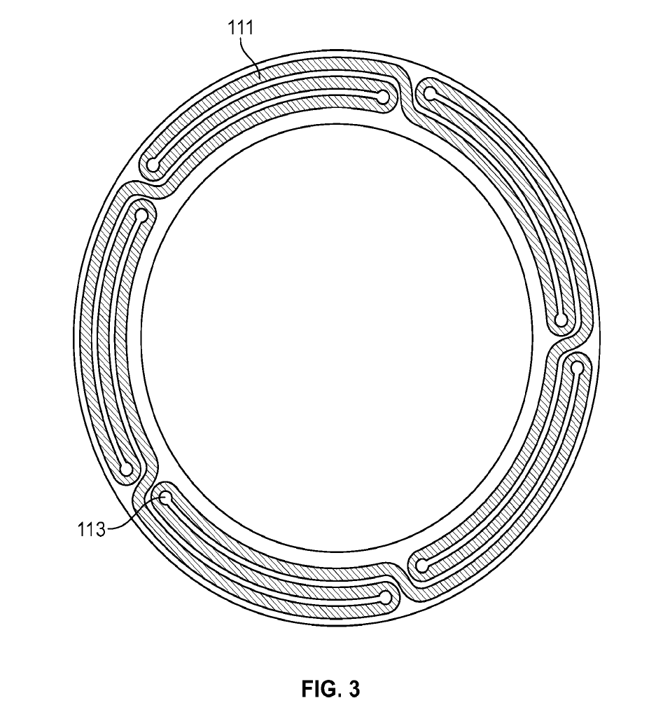

Likewise, referring now to FIG. 2 and to FIG. 3, reinforcing struts 111 expand at inflation to unwrap balloon 101 evenly from proximal end 103. Folding points 111 enable reinforcing struts 111 to maintain a minimum profile of system 100.

Referring now to FIG. 3, pre-stressed struts 111 are functionally reinforcing during folding/unfolding. Said pre-stressed struts 111 function as reinforcing struts and return to folding points 113 upon retraction into delivery sheaths, as known to artisans. This, lower French-scale sizing is achieved over conventional PTA balloon systems.

While the method and apparatus have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

It should also be understood that a variety of changes may be made without departing from the essence of the invention. Such changes are also implicitly included in the description. They still fall within the scope of this invention. It should be understood that this disclosure is intended to yield a patent covering numerous aspects of the invention both independently and as an overall system and in both method and apparatus modes.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these.

Particularly, it should be understood that as the disclosure relates to elements of the invention, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

Finally, all references listed in the Information Disclosure Statement or other information statement filed with the application are hereby appended and hereby incorporated by reference; however, as to each of the above, to the extent that such information or statements incorporated by reference might be considered inconsistent with the patenting of this/these invention(s), such statements are expressly not to be considered as made by the applicant.

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

1. A medical device system, comprising, in combination:
a pressurized balloon adopted for inflation and deflation between an expanded diameter and a collapsed configuration;
wherein mounting of the balloon on a catheter system is defined by a spiral wrap of the balloon during the collapsed configuration; and,
the balloon is equipped with struts for reinforcement, whereby the struts are at fold points and aid in the spiral folding and unfolding, stabilized and consistent inflation and deflation of the balloon.

2. The system of claim 1, wherein said pre-stressed struts provide for increased radiopacity, without increasing profile of a pre-inserted catheter assembly.

3. The system of claim 2, wherein said balloon is high pressure and has a lower profile, on the French scale of measurement, than balloons having similar burst resistance rating.

4. The system of claim 3, burst pressure ranging from at least about 18 to approximately 22 atm.

5. The system of claim 3, burst pressure ranging from at least about 20 to approximately 25 atm.

6. The system of claim 3, burst pressure ranging from at least about 26 to approximately 30 atm.

7. The system of claim 3, burst pressure ranging from at least about 30 to approximately 40 atm.
